(19) United States
(12) Patent Application Publication
(10) Pub. No.: US 2022/0062362 A1
(43) Pub. Date: Mar. 3, 2022

(54) Composition for Increasing Occupancy of Faecalibacterium in Intestinal Flora

(71) Applicant: Sumitomo Chemical Company, Limited, Tokyo (JP)

(72) Inventors: Akihiro Yamamori, Ibaraki (JP); Katsunori Sasaki, Tokyo (JP)

(73) Assignee: Sumitomo Chemical Company, Limited, Tokyo (JP)

(21) Appl. No.: 17/435,568

(22) PCT Filed: Mar. 4, 2020
(86) PCT No.: PCT/JP2020/009067

§ 371 Date: Sep. 1, 2021

(30) Foreign Application Priority Data
Mar. 5, 2019 (JP) 2019-039870

(51) Int. Cl. A61K 36/258; A61K 36/9068; A61K 36/758

(52) U.S. Cl. CPC A61K 36/258; C12N 1/205; A61K 36/758; A61K 36/9068

(57) ABSTRACT

Disclosed is a composition for increasing the occupancy of bacteria of the genus Faecalibacterium in the intestinal flora, the composition comprising a Daikenchuto extract.

### TECHNICAL FIELD

The present invention relates to a composition for increasing the occupancy of bacteria of the genus Faecalibacterium in the intestinal flora.

### BACKGROUND ART

The bacteria of the genus Faecalibacterium are short-chain fatty acid-producing bacteria that mainly produce butyric acid as a metabolite. Butyric acid, which has an ability to induce regulatory T cells (Treg), has been found to have anti-inflammatory and anti-allergic effects. The occupancy of bacteria of the genus Faecalibacterium in the human intestinal flora is as high as a few percent, and it is indicated that a decrease in bacteria of the genus Faecalibacterium might be associated with Crohn's disease etc. (Non-patent Literature (NPL) 1).

Cacao has conventionally been known to contribute to the increase in the occupancy of bacteria of the genus Faecalibacterium. For example, Patent Literature (PTL) 1 reports that processed oil and fat compositions containing a high concentration of cacao bean-derived luminacoid significantly increase the occupancy of bacteria of the genus Faecalibacterium in the human intestinal flora.

However, cacao contains theobromine, caffeine, etc. Since theobromine and caffeine have, for example, bronchodilator, diuretic, and stimulant effects, people who are sensitive to these components and people who are taking medicines such as theophylline, as well as pregnant women etc., should be careful when consuming food products with a high cacao content.

### CITATION LIST

### Patent Literature

PTL 1: WO 2018/056284

### Non-Patent Literature

NPL 1: The ISME Journal (2017) 11, 841-852

SUMMARY OF INVENTION

### Technical Problem

An object of the present invention is to provide a composition for increasing the occupancy of bacteria of the genus Faecalibacterium.

### Solution to Problem

The present inventor conducted extensive research to solve the above problem, and consequently found that a Daikenchuto extract, which is a drug substance of Chinese medicine, is capable of significantly increasing the occupancy (%) of the bacteria of the genus Faecalibacterium in the human intestinal flora.

The present invention has been completed as a result of further research based on the above findings. The present invention provides the following compositions for increasing the occupancy of bacteria of the genus Faecalibacterium in the intestinal flora.
Item 1. A composition for increasing the occupancy of bacteria of the genus Faecalibacterium in the intestinal flora, the composition comprising a Daikenchuto extract.
Item 2. The composition according to Item 1, wherein the Daikenchuto extract is contained in a daily intake amount effective to increase the occupancy of bacteria of the genus Faecalibacterium in the intestinal flora.
Item 3. The composition according to Item 1 or 2, wherein the Daikenchuto extract is contained in a daily intake amount of 0.1 to 15 g.
Item 4. The composition according to Item 1 or 2, wherein the Daikenchuto extract is contained in a daily intake amount of 0.1 to 3.0 g.
Item 5. The composition according to any one of Items 1 to 4, wherein the mass ratio of crude drug raw materials for use in obtaining the Daikenchuto extract is such that Zanthoxylum fruit or Zanthoxylum bungeanum:ginseng:Zingiber siccatum is 1:0.2 to 2.0:1.0 to 3.0.
Item 6. The composition according to any one of Items 1 to 4, wherein the mass ratio of crude drug raw materials for use in obtaining the Daikenchuto extract is such that Zanthoxylum fruit or Zanthoxylum bungeanum:ginseng:Zingiber siccatum is 1:0.4 to 1.4:1.4 to 2.4.
Item 7. The composition according to any one of Items 1 to 6 for use in treating, preventing, or ameliorating a disease or symptom that can be treated, prevented, or ameliorated by an increase in the occupancy of bacteria of the genus Faecalibacterium in the intestinal flora.
Item 8. The composition according to any one of Items 1 to 7, which is a food product.

The present invention also provides the following.
Item 9. A method for increasing the occupancy of bacteria of the genus Faecalibacterium in the intestinal flora, the method comprising administering a composition comprising a Daikenchuto extract to a mammal in need thereof.
Item 10. Use of a Daikenchuto extract in the manufacture of a composition for increasing the occupancy of bacteria of the genus Faecalibacterium in the intestinal flora.
Item 11. The method according to Item 9, or the use according to Item 10, wherein the composition comprises the Daikenchuto extract in a daily intake amount effective to increase the occupancy of bacteria of the genus Faecalibacterium in the intestinal flora.
Item 12. The method according to Item 9 or 11, or the use according to Item 10 or 11, wherein the composition comprises the Daikenchuto extract in a daily intake amount of 0.1 to 15 g.
Item 13. The method according to Item 9 or 11, or the use according to Item 10 or 11, wherein the composition comprises the Daikenchuto extract in a daily intake amount of 0.1 to 3.0 g.
Item 14. The method according to any one of Items 9 and 11 to 13, or the use according to any one of Items 10 to 13, wherein the mass ratio of crude drug raw materials for use in obtaining the Daikenchuto extract is such that Zanthoxylum fruit or Zanthoxylum bungeanum:ginseng:Zingiber siccatum is 1:0.2 to 2.0:1.0 to 3.0
Item 15. The method according to any one of Items 9 and 11 to 13, or the use according to any one of Items 10 to 13, wherein the mass ratio of crude drug raw materials for use in obtaining the Daikenchuto extract is such that Zanthoxylum fruit or Zanthoxylum bungeanum:ginseng:Zingiber siccatum is 1:0.4 to 1.4:1.4 to 2.4.
Item 16. The method according to any one of Items 9 and 11 to 15, or the use according to any one of Items 10 to 15, wherein the composition is a food product.
Item 17. The method according to any one of Items 9 and 11 to 16, which is a method for treating, preventing, or ameliorating a disease or symptom capable of being treated, prevented, or ameliorated by an increase in the occupancy of bacteria of the genus Faecalibacterium in the intestinal flora.
Item 18. The use according to any one of Items 10 to 16, wherein the composition is used for treating, preventing, or ameliorating a disease or symptom that can be treated, prevented, or ameliorated by an increase in the occupancy of bacteria of the genus Faecalibacterium in the intestinal flora.

### Advantageous Effects of Invention

The composition of the present invention is capable of effectively increasing the occupancy of bacteria of the genus Faecalibacterium in the intestinal flora. In particular, the composition of the present invention is capable of increasing the occupancy of bacteria of the genus Faecalibacterium in the adult body, without using theobromine or caffeine.

The composition of the present invention, which increases the occupancy of bacteria of the genus Faecalibacterium in the intestinal flora, can contribute to the prevention and treatment of diseases or symptoms that can be treated, prevented, or ameliorated by an increase in the occupancy of bacteria of the genus Faecalibacterium.

### BRIEF DESCRIPTION OF DRAWINGS

FIG. 1 is a graph showing the occupancy of bacteria of the genus Faecalibacterium in bacterial flora before and after ingestion of test food in each subject.

### DESCRIPTION OF EMBODIMENTS

The prevent invention is described in detail below.

In the present specification, the term “comprise” also includes the meanings of “essentially consist of” and “consist of.”

The composition of the present invention is for increasing the occupancy of bacteria of the genus Faecalibacterium in the intestinal flora, and is characterized by comprising a Daikenchuto extract.

Bacteria of the genus Faecalibacterium are microorganisms that belong to the Clostridium cluster IV (C. leptum subgroup), and that belong to the genus Faecalibacterium. Examples of microorganisms that belong to the genus Faecalibacterium include Faecalibacterium prausnitzii.

Intestinal microbiota (intestinal flora) refers to populations of bacteria present in the intestines, and is also called gut flora and gut microbiota.

Daikenchuto is described in “Jingui Yaolüe,” a clinical book from the Chinese Song Dynasty. It is considered to have, for example, effects of enhancing intestinal motility, inhibiting excessive intestinal motility, inhibiting ileus, and increasing the intestinal blood flow, as well as anti-inflammatory effects. Daikenchuto is usually obtained by using an extract in a powder form of a crude drug mixture consisting of 2.0 g of Zanthoxylum fruit or Zanthoxylum bungeanum, 3.0 g of ginseng, and 5.0 g of Zingiber siccatum (sometimes also called Mukoi-Daikenchuto), to which a powdered candy (Saccharum granorum (koi), maltose) in an amount that is about 8 times the amount of the extract is blended. Daikenchuto is usually formulated from Zanthoxylum fruit or Zanthoxylum bungeanum:ginseng:Zingiber siccatum:Saccharum granorum in a mass ratio of 2.0:3.0:5.0:20.

In the present specification, the term “Daikenchuto extract” refers to an extract of a crude drug mixture that does not contain Saccharum granorum. However, the Daikenchuto extract contained in the composition of the present invention may be in the form of the extract itself, or in the form a mixture of the extract with Saccharum granorum.

The composition of the present invention may consist only of the Daikenchuto extract. Alternatively, the composition of the present invention may comprise additional components, in addition to the Daikenchuto extract. The amount of the Daikenchuto extract contained in the composition of the present invention may be appropriately set according to the form, dosage form, symptoms, etc. When additional components are contained, the proportion of the Daikenchuto extract in the composition of the present invention is not particularly limited; it is typically, for example, 0.1 to 99.9 mass %, preferably 1 to 50 mass %, and more preferably 4 to 30 mass %.

The composition of the present invention can be in any form, such as jelly, powder, granule, tablet, capsule, liquid, suspension, or emulsion form.

The composition of the present invention is preferably administered by oral administration. Administration methods other than oral administration are also applicable. For example, a base may be incorporated in the composition of the present invention for the purpose of efficient transportation to the intestines. The base may be a general-purpose gelling agent. Since appropriate storage stability is required, a gelling agent other than gelatin is preferred. Examples of such gelling agents include carrageenan, locust bean gum, xanthan gum, polyacrylic acid, and carob bean gum.

In the present invention, the phrase “increasing the occupancy of bacteria of the genus Faecalibacterium in the intestinal flora” means increasing the occupancy of bacteria of the genus Faecalibacterium in the intestinal flora after ingestion or administration of the composition of the present invention by, for example, 1% or more, 2% or more, 3% or more, 4% or more, 5% or more, 6% or more, 7% or more, 8% or more, 9% or more, or 10% or more, compared to before ingestion or administration.

The composition of the present invention can be used for therapeutic or non-therapeutic applications. Further, the composition of the present invention is applicable, in particular, to mammals including humans (e.g., humans, monkeys, mice, rats, rabbits, dogs, cats, horses, cows, and pigs; preferably humans).

The composition of the present invention may be used in the treatment, prevention, or amelioration of diseases or symptoms that are effectively treated, prevented, or ameliorated by an increase in the occupancy of bacteria of the genus Faecalibacterium in the intestinal flora.

Examples of the diseases and symptoms that can be treated, prevented, or ameliorated by an increase in the occupancy of the bacteria of the genus Faecalibacterium include inflammatory diseases (e.g., ulcerative colitis, Crohn's disease, and other inflammatory bowel diseases), allergic diseases (e.g., food allergies, hay fever, asthma, allergic rhinitis, drug allergies, atopic dermatitis, and mite allergies), and diseases believed to be caused by dysbiosis (e.g., Crohn's disease, inflammatory bowel diseases, irritable bowel syndrome, small intestinal bacterial overgrowth (SIBO), pseudomembranous enterocolitis (Clostridium difficile colitis), colorectal cancer, antimicrobial-induced diarrhea, liver diseases, such as alcoholic steatohepatitis (ASH) and nonalcoholic steatohepatitis (NASH), type 2 diabetes, obesity, allergic diseases, mental disorders such as autism, and multiple sclerosis).

The composition of the present invention can be used as food products, pharmaceuticals, quasi-drugs, feeds, and the like. The composition of the present invention also encompasses the meanings of additives for foods, pharmaceuticals, quasi-drugs, feeds, and the like.

In the present specification, the term “food product” also encompasses the meanings of food and beverages for the purpose of health, health maintenance, health promotion, etc. (e.g., health food, functional food, dietary supplement, supplement, food for specified health uses, food with nutrient function, or food with functional claims), food for infants, food for pregnant and nursing women, food for the sick, and the like.

Although the Daikenchuto extract can be used as is in a food product, it is also possible to add additional components, if necessary.

The food products include any food products and beverages that can be consumed by animals (including humans). The type of food products is not particularly limited. Examples include dairy products; fermented food products (e.g., yogurt and cheese); beverages (e.g., soft drinks such as coffee, juice, and tea beverages, carbonated beverages, milk beverages, lactic acid bacteria beverages, lactic acid bacteria-containing beverages, yogurt beverages, and liquors such as Japanese sake, fruit wine, and Western liquors); spreads (e.g., custard cream); pastes (e.g., fruit pastes); western confectioneries (e.g., donuts, pies, cream puffs, gum, candies, jellies, cookies, cakes, chocolates, and puddings); Japanese sweets (e.g., daifuku, mochi, manju, castella, anmitsu, and yokan); frozen desserts (e.g., ice cream, ice pops, and sherbet); food products (e.g., curry, beef bowl, rice porridge, miso soup, soup, meat sauce, pasta, pickles, and jam); and seasonings (e.g., dressings, flavor enhancers, sprinkled flavorings for rice, and soup ingredients).

The production method for the food products is also not particularly limited, and can follow known methods as appropriate.

The dosage unit form when using the food product as a supplement is not particularly limited, and can be appropriately selected. Examples include tablets, capsules, granules, liquids, sprays, and jellies.

The amount of intake of the food product can be appropriately set according to various conditions such as the body weight, age, gender, and symptoms of the person taking the food product. The daily intake amount of the Daikenchuto extract is, for example, stated later.

To prepare pharmaceuticals and quasi-drugs, the Daikenchuto extract can be used as is. Alternatively, the Daikenchuto extract can be used with a non-toxic carrier, diluent, or excipient acceptable in pharmaceuticals to obtain preparations for pharmaceutical use in the form of, for example, tablets (e.g., uncoated tablets, sugar-coated tablets, film-coated tablets, foam tablets, chewable tablets, and lozenges), capsules, pills, powders (sprays), fine granules, granules, liquids, suspensions, emulsions, jellies, syrups, and pastes. Examples of the non-toxic carriers acceptable in pharmaceuticals include binders, additives, flavors, buffers, thickeners, colorants, stabilizers, emulsifiers, dispersants, suspending agents, and preservatives.

The administration method for the pharmaceuticals and quasi-drugs is not particularly limited. Examples include oral administration, rectal administration, enteral administration, buccal administration, intra-arterial administration, intravenous administration, and transdermal administration.

The administration amount of the pharmaceuticals and quasi-drugs may be appropriately determined according to various conditions such as the body weight, age, gender, and symptoms of the patient. The daily administration amount of the Daikenchuto extract, for example, is stated later.

Several Daikenchuto extracts are already available for the market as pharmaceutical products. However, from the standpoint of availability, the composition of the present invention is preferably used as a food product.

The food products, pharmaceuticals, quasi-drugs, etc., may be provided with a label stating that, for example, the product is for increasing the occupancy of bacteria of the genus Faecalibacterium in the intestinal flora, or for expanding the occupancy of butyric acid-producing bacteria in the intestinal flora.

The composition of the present invention may also be used in combination with other compositions. For example, a combined use of the composition of the present invention with materials and compositions that are expected to exhibit an effect of increasing the occupancy of bacteria of the genus Faecalibacterium can further improve the effect of increasing the occupancy of bacteria of the genus Faecalibacterium in the intestinal flora.

In the composition of the present invention, the dose of the Daikenchuto extract must be an appropriate amount because an overly high dose will lead to diarrhea, and an overly low dose will not provide the desired effect. The daily dose (daily intake amount) of the Daikenchuto extract is preferably 0.1 to 15 g of an extract of 0.5 to 6 g of Zanthoxylum fruit or Zanthoxylum bungeanum, 0.5 to 6 g of ginseng, and 1 to 12 g of Zingiber siccatum, which are crude drugs; more preferably 0.5 to 6 g of an extract of 0.5 to 2 g of Zanthoxylum bungeanum, 0.5 to 2 g of ginseng, and 1 to 4 g of Zingiber siccatum; and still more preferably 1 to 2 g of an extract of 1 to 1.5 g of Zanthoxylum bungeanum, 1 to 1.5 g of ginseng, and 1.5 to 2.5 g of Zingiber siccatum. The dose is not limited to a once-daily dose, and can be divided into multiple daily doses.

The mass ratio of the crude drug raw materials for use in obtaining the Daikenchuto extract contained in the composition of the present invention is preferably such that Zanthoxylum fruit or Zanthoxylum bungeanum:ginseng:Zingiber siccatum is 1:0.2 to 2.0:1.0 to 3.0, more preferably 1:0.4 to 1.4:1.4 to 2.4, and still more preferably 1:0.6 to 1.2:1.6 to 2.2.

The results of the Examples described below confirmed that the composition of the present invention can be taken for 7 continuous days, and confirmed an increase in the occupancy of the genus Faecalibacterium 7 days later. Therefore, the composition of the present invention is preferably ingested continuously for one week or more, more preferably two weeks or more, and still more preferably three weeks or more.

Daikenchuto promotes intestinal motility, which can cause a rumbling sensation in the stomach or the passing of gas after ingestion. Therefore, ingestion is preferably carried out before bedtime. As shown in the Examples described below, ingestion before bedtime showed actual achievement.

The composition of the present invention may comprise the Daikenchuto extract in a daily intake amount effective to increase the occupancy of bacteria of the genus Faecalibacterium in the intestinal flora. In this case, the composition of the present invention may be packaged to provide an effective daily intake amount. The packaging form may be a single package or multiple packages, as long as the effective daily intake amount can be provided. When the effective daily intake amount is divided into multiple packages, the multiple packages containing the effective daily intake amount can be used as a set. For example, the effective daily intake amount of the Daikenchuto extract is, for example, 0.1 to 15 g, preferably 0.1 to 3.0 g, and more preferably 0.5 to 2.0 g.

The packaging form is not particularly limited as long as a predetermined amount of the extract can be contained. Examples include bags, wrapping paper, paper containers, soft packs, cans, bottles, and capsules.

The composition of the present invention is capable of effectively increasing the occupancy of bacteria of the genus Faecalibacterium in the intestinal flora. In particular, the composition of the present invention is capable of increasing the occupancy of bacteria of the genus Faecalibacterium in the human body within a short period of time, without using theobromine or caffeine. In addition, the composition of the present invention, which increases the occupancy of bacteria of the genus Faecalibacterium in the intestinal flora, can contribute to the prevention and treatment of diseases or symptoms that can be treated, prevented, or ameliorated by an increase in the occupancy of bacteria of the genus Faecalibacterium.

### Examples

The present invention is described in further detail below with reference to Examples. However, the present invention is not limited to these Examples etc.

### Human Study

### Preparation of Composition

A Daikenchuto extract was extracted by using commercially available Zanthoxylum bungeanum, Asian ginseng, and Zingiber siccatum in a mass ratio of 1:1:2 by using a known method. Then, 1.25 g of the obtained extract, 2.6 g of syrup, 0.085 g of ethanol, and 0.565 g of purified water were mixed to give 4.5 g of a composition (daily intake amount).

### Human Study

Five healthy subjects between the ages of 20 or older and younger than 60 were selected by screening, and the composition was ingested before bedtime on day 1 to day 7. Water or hot water may be taken at the same time as ingesting the composition. The occupancy in the bacterial flora was measured on the day before the ingestion of the composition (day 0) and the morning of the next day after 7 days of the ingestion (day 8).

### Lifestyle During the Test

The subjects were instructed to lead life as usual during the test period, and obey the following restrictions.
Do not drastically change eating habits (amount and content of food), and refrain from an overly strict dietary restriction, as well as overeating, which would greatly deviate from a daily routine.
Refrain from drastically changing the usual level of alcohol consumption; however, alcohol consumption is not restricted as a general rule.
Refrain as much as possible from making any major life changes until the stool collection one week after the ingestion is completed.

### Bacterial Flora Measurement Method

DNA in feces was purified, and the 16S rRNA V1-V2 region was amplified for sequence analysis with a MiSeq next-generation sequencer (Illumina). After quality filtering, 10,000 reads were randomly selected from each sample, clustered at 97% homology, and classified into operational taxonomic units (OTUs). The representative sequences of the formed OTUs were collated from the full-length 16S gene database to identify the species and analyze the composition. The QIIME pipeline was used in the process from clustering to bacterial flora composition analysis while Greengenes was used for the full-length 16S gene database.

### REFERENCES

QIIME: Nat Methods. 2010 May; 7(5): 335-6. DOI: 10.1038/nmeth.f.303. Epub 2010 Apr. 11.
Greengenes: Applied and Environmental Microbiology, July 2006, Vol. 72, No. 7, pp. 5069-5072, DOI: 10.1128/AEM.03006-05

### Results

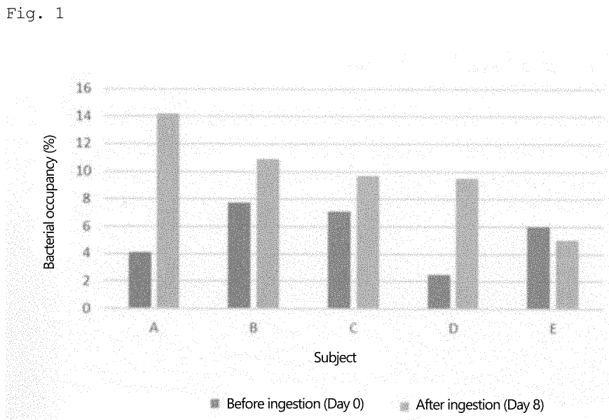

Table 1 and FIG. 1 show the results. In the intestinal flora in the subjects (genus level), the occupancy of bacteria of the genus Faecalibacterium increased by 2% or more in 4 out of 5 subjects in the test food ingestion group after ingestion (day 8) compared to before ingestion (day 0), with the total bacterial count in the intestinal flora taken as 100% (Table 1, FIG. 1).

TABLE 1

Occupancy of the genus Faecalibacterium in
bacterial flora (%)

| | Day 0 | Day 8 |
|---|---|---|
| Subject A | 4.1 | 14.2 |
| Subject B | 7.7 | 10.9 |
| Subject C | 7.1 | 9.7 |
| Subject D | 2.5 | 9.5 |
| Subject E | 6.0 | 5.0 |

1-8. (canceled)

9. A method for increasing the occupancy of bacteria of the genus Faecalibacterium in the intestinal flora, the method comprising administering a composition comprising a Daikenchuto extract to a mammal in need thereof.

10. The method according to claim 9, wherein the composition comprises the Daikenchuto extract in a daily intake amount effective to increase the occupancy of bacteria of the genus Faecalibacterium in the intestinal flora.

11. The method according to claim 9, wherein the composition comprises the Daikenchuto extract in a daily intake amount of 0.1 to 15 g.

12. The method according to claim 9, wherein the composition comprises the Daikenchuto extract in a daily intake amount of 0.1 to 3.0 g.

13. The method according to claim 9, wherein the mass ratio of crude drug raw materials for use in obtaining the Daikenchuto extract is such that Zanthoxylum fruit or Zanthoxylum bungeanum:ginseng:Zingiber siccatum is 1:0.2 to 2.0:1.0 to 3.0

14. The method according to claim 9, wherein the mass ratio of crude drug raw materials for use in obtaining the Daikenchuto extract is such that Zanthoxylum fruit or Zanthoxylum bungeanum:ginseng:Zingiber siccatum is 1:0.4 to 1.4:1.4 to 2.4.

15. The method according to claim 9, wherein the composition is a food product.

16. The method according to claim 9, which is a method for treating, preventing, or ameliorating a disease or symptom capable of being treated, prevented, or ameliorated by an increase in the occupancy of bacteria of the genus Faecalibacterium in the intestinal flora.
